(19) United States
(12) Patent Application Publication
(10) Pub. No.: US 2020/0319125 A1
(43) Pub. Date: Oct. 8, 2020

(54) SIMULATION METHOD, SIMULATION PROGRAM, AND SIMULATION DEVICE INCLUDING STORAGE MEDIUM HAVING SAID PROGRAM STORED THEREIN

(71) Applicant: Hisaka Works, Ltd., Osaka-shi, Osaka (JP)

(72) Inventors: Hirokazu Negoro, Higashi-Osaka-shi, Osaka (JP); Isamu Mukai, Amagasaki-shi, Hyogo (JP); Kota Ukai, Higashi-Osaka-shi, Osaka (JP)

(21) Appl. No.: 16/305,331

(22) PCT Filed: May 17, 2017
(86) PCT No.: PCT/JP2017/018449

§ 371 Date: Nov. 28, 2018

(30) Foreign Application Priority Data
May 31, 2016 (JP) 2016-108863

(51) Int. Cl. G01N 25/18; A23L 3/10; A61L 2/24; A61L 2/04; A61L 2/00

(52) U.S. Cl. CPC G01N 25/18; A23L 3/10; A61L 2/24; A61L 2202/21; A61L 2/0023; A23V 2002/00; A61L 2202/24; A61L 2/04

(57) ABSTRACT

A simulation method includes: a step of setting a heating condition of an object; a step of calculating a first estimated temperature as a temperature of a central portion of the object, based on the heating condition; and a step of calculating a second estimated temperature as a temperature in a second area that surrounds a first area positioned in the central portion of the object, based on the first estimated temperature.

### CROSS-REFERENCE TO RELATED APPLICATION

This application claims priority to Japanese Patent Application No. 2016-108863, the disclosure of which is incorporated herein by reference in its entirety.

### FIELD

The present invention relates to a simulation method for calculating temperature change of an object that is being heated, a simulation program, and a simulator including a storage medium having the program stored therein.

### BACKGROUND

In manufacturing packed food, such as canned food and retort food, the manufactured packed food is conventionally heat sterilized. Whether or not a certain heating condition is suitable for food sterilization is generally evaluated using an F-value, which is a sterilization value expressed in a relation between temperature and time. In the case where the temperature history of food under a certain heating condition meets a predetermined F-value, it can be evaluated that the heating condition is suitable for food sterilization. For example, an F-value of retort food is required to be equivalent to 4 minutes at 120.0° C. or greater, pursuant to the Food Sanitation Act.

The temperature history of food can also be obtained through measurements of the temperature of the food that is being heated using a sensor or the like; however, it requires costs and time to obtain the temperature histories of foods having different shapes and sizes through the measurements. In order to reduce these costs and time, a simulation method using the computer for calculating an estimated temperature of food that is being heated is widely used (see, for example, Patent Literature 1).

### CITATION LIST

### Patent Literature

Patent Literature 1; JP 3071412 B

Based on the premise that food temperature rises with heat transfer from the atmosphere, a central portion of food is least likely to receive heat and least likely to be sterilized. Thus, in the case where heating conditions are evaluated using a conventional simulation method, an estimated temperature of the central portion of food is used as an index for evaluating the heating conditions. Note that the central portion in this context does not refer to the physical center of the object but to a portion in which a rise or fall in temperature occurs lastly.

Heating of food affects not only sterilization of food but also degradation of proteins or vitamins present in food. The same applies to heating of an object other than food. For example, heating of a medicinal product or a medical device may have an adverse effect on, for example, the components of the medicinal product or on the materials forming the medical device. It is thus required to evaluate the heating conditions of an object to be processed also from other viewpoints than sterilization.

### SUMMARY

### Technical Problem

In view of the above circumstances, the present invention is to provide a simulation method capable of evaluating a heating condition of an object not only from the viewpoint of sterilization thereof but also from other viewpoints than sterilization.

### Solution to Problem

The simulation method according to the present invention includes: a step of setting a heating condition of an object; a step of calculating a first estimated temperature as a temperature of a central portion of the object, based on the heating condition; and a step of calculating a second estimated temperature as a temperature in a second area that surrounds a first area positioned in the central portion of the object, based on the first estimated temperature.

As one aspect of the simulation method according to the present invention, the simulation method may further include a step of evaluating a temperature history of the object, based on the first estimated temperature, the second estimated temperature, a ratio of a volume of the first area to a volume of the object, and a ratio of a volume of the second area to the volume of the object.

As one aspect of the simulation method according to the present invention, in the step of evaluating the temperature history of the object, a third estimated temperature may be calculated based on the first estimated temperature, the second estimated temperature, the ratio of the volume of the first area to the volume of the object, and the ratio of the volume of the second area to the volume of the object, to evaluate the temperature history of the object using the third estimated temperature.

As one aspect of the simulation method according to the present invention, the heating condition may include an ambient temperature and a heating time of the object, and the second estimated temperature may be an average of the first estimated temperature of the object and the ambient temperature.

The simulation program according to the present invention is a simulation program for causing an arithmetic unit to execute calculation of an estimated temperature of an object to be heated, the simulation program causing the arithmetic unit to execute: a step of receiving a setting of a heating condition of the object; a step of calculating a first estimated temperature as a temperature of a central portion of the object, based on the heating condition; and a step of calculating a second estimated temperature as a temperature in a second area that surrounds a first area positioned in the central portion of the object, based on the first estimated temperature.

The simulator according to the present invention includes a storage medium having the simulation program stored therein, and is configured to cause the arithmetic unit to run the simulation program.

According to the present invention, the simulation method capable of evaluating a heating condition not only from the viewpoint of sterilization of the object but also from other viewpoints than sterilization can be provided.

### BRIEF DESCRIPTION OF DRAWINGS

FIG. 1 is a flow chart of a simulation method according to an embodiment of the present invention.

FIG. 2 is a model diagram of an object of which an estimated temperature is calculated using the simulation method according to an embodiment of the present invention.

FIG. 3 is a model diagram of an object of which an estimated temperature is calculated using the simulation method according to another embodiment of the present invention.

### DESCRIPTION OF EMBODIMENTS

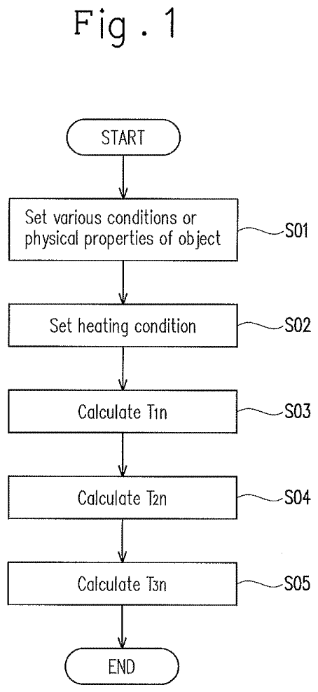

Hereinafter, the simulation method of the present invention will be described with reference to the attached drawings. As shown in the flow chart of FIG. 1, the simulation method of this embodiment includes: a step (S01) of setting various conditions or physical properties of an object to a simulator; a step (S02) of setting a heating condition to the simulator; a step (S03) of causing the simulator to calculate a first estimated temperature T₁ₙ; a step (S04) of causing the simulator to calculate a second estimated temperature T₂ₙ; and a step (S05) of causing the simulator to calculate a third estimated temperature T₃ₙ. The simulation method of this embodiment is performed by the simulator that includes a storage medium in which a program capable of executing the simulation method is stored, and an arithmetic unit (CPU) for running the program. A result of the simulation is, for example, displayed on a display provided to the simulator, and stored in a memory card. Hereinafter, each of the steps included in the simulation method of this embodiment will be described in order. Note that this embodiment will be described by taking, for example, the case where the ATS method (Ambient Temperature Slide method) is employed as a method for calculating the temperature of a central portion of the object.

First, a summary of the ATS method will be described. It is premised in the ATS method that the temperature of the object rises with heat transfer from the atmosphere. Calculations are made based on the premise that the “amount of heat the atmosphere transfers to the object” equals the “amount of heat the object receives from the atmosphere”. Where a unit time is Δt, a central point of the object is a central point P, an ambient temperature and a central point temperature of the object are respectively T_{wn} and Tₚₙ (the subscript n represents the nth interval in intervals of time), a surface temperature of the object equals the ambient temperature T_{wn}, a linear temperature gradient is caused inside the object, the distance from the surface of the object to the central point is L, the thermal conductivity of the object is k, and the surface area of the object is A, the “amount of heat the atmosphere transfers to the object” per unit time, i.e., the left side of the formula below, is obtained.

In addition, where the volume of the object is V, the density of the object is p, the specific heat of the object is cₚ, and a central point temperature Tₚₙ equals a volume average temperature Tₚ*ₙ, the “amount of heat the object receives from the atmosphere” per unit time, i.e., the right side of the formula below, is obtained:

kA(T_{wn-1}−Tₚₙ₋₁)Δt/L=Vρcₚ(Tₚ*ₙ−Tₚ*ₙ₋₁)

When the formula above is arranged assuming that k/(ρcₚ) is a thermal diffusivity coefficient α, the formula below is given:

Tₚ*ₙ=Tₚ*ₙ₋₁+αΔt(T_{wn-1}−Tₚₙ₋₁)/L²

Since the central point temperature Tₚₙ actually differs from the volume average temperature Tₚ*ₙ, a deviation ratio β between the central point temperature Tₚₙ and the volume average temperature Tₚ*ₙ is used to make Tₚₙ=Tₚ*nβ, so that the formula below is given:

Tₚₙ=Tₚₙ₋₁+αβΔt(T_{wn-1}−Tₚₙ₋₁)/L²

Further, where αβΔt/L² is a heat transfer coefficient τ, the formula below is given. The formula below expresses that rise in the central point temperature corresponds to the product of multiplication of the heat transfer coefficient τ and the difference between a surface temperature T_{wn-1} and a central point temperature Tₚₙ₋₁, of the object, per unit time:

Tₚₙ=Tₚₙ₋₁+τ(T_{wn-1}−Tₚₙ₋₁)

In the actual object, however, the surface thereof is located away from the central point; thus, temperature change of the central point is delayed compared to temperature change of the surface. This delay is not reflected in the formula above. Therefore, in the formula above, a virtual ambient temperature T_{w}^{†}tₙ₋₁ obtained by delaying temporal change of an ambient temperature T_{wn-1} by δ₁ is used as the surface temperature instead of the ambient temperature T_{wn-1}, so that the formula below is given:

Tₚₙ=Tₚₙ₋₁+τ(T_{w}^{†}ₙ₋₁−Tₚₙ₋₁)

Thus, in the case where the ATS method is employed, the heat transfer coefficient τ and a delay time δ₁ are set to the simulator by, for example, a user's input in step S01. The heat transfer coefficient τ and the delay time δ₁ vary by a material forming the object and a shape of the object. The user inputs the data of the heat transfer coefficient τ and the delay time δ₁ to the simulator if the user has the data. If the user does not have the data of the heat transfer coefficient τ and the delay time δ₁, the user obtains the heat transfer coefficient τ and the delay time δ₁, and inputs the data to the simulator. To obtain the heat transfer coefficient τ and the delay time δ₁, the user estimates an arbitrary heat transfer coefficient τ and an arbitrary delay time δ₁, calculates the first estimated temperature T₁ₙ using the ATS method based on the estimated values, compares the first estimated temperature T₁ₙ with the measured temperature to adjust the estimated heat transfer coefficient τ and the estimated delay time δ₁, recalculates the first estimated temperature T₁ₙ based on the adjusted heat transfer coefficient τ and the adjusted delay time δ₁, and repeatedly performs these until the first estimated temperature T₁ₙ is approximated to the measured temperature. In this way, the appropriate heat transfer coefficient τ and the appropriate delay time δ₁ can be obtained. That is, a trial-and-error method can be employed. The measured temperature of the object is measured using a temperature detecting sensor such as a thermistor.

When, for example, the heat transfer coefficient τ is unknown, a first estimated temperature T′₁ₙ is calculated based on an estimated heat transfer coefficient τ′₁ to calculate the difference between the first estimated temperature T′₁ₙ and the measured temperature. When the difference between the first estimated temperature T′₁ₙ and the measured temperature is equal to or less than a desired value, the heat transfer coefficient τ′₁ is input to the simulator.

When the difference between the first estimated temperature T′₁ₙ and the measured temperature is greater than the desired value, four heat transfer coefficients t″₁ close to the heat transfer coefficient t′₁ are estimated to calculate first estimated temperatures T″₁ₙ respectively based on the four heat transfer coefficients τ″₁ and calculate the difference between each of the four first estimated temperatures T″₁ₙ and the measured temperature.

When the smallest one of the differences between the four first estimated temperatures T″₁ₙ and the measured temperature is smaller than the difference between the first estimated temperature T′₁ₙ and the measured temperature and is smaller than the desired value, then the heat transfer coefficient t″₁ used for calculating the smallest one of the differences between the four first estimated temperatures T″₁ₙ and the measured temperature is input to the simulator.

When the smallest one of the differences between the four first estimated temperatures T″₁ₙ and the measured temperature is smaller than the difference between the first estimated temperature T′₁ₙ and the measured temperature and is greater than the desired value, then four heat transfer coefficients τ′″₁ that are close to the heat transfer coefficient τ′₁ used for calculating the smallest one of the differences between the four first estimated temperatures T″₁ₙ and the measured temperature are estimated to repeatedly carry out a series of calculations, such as calculations of first estimated temperatures T′″₁ₙ respectively based on the four heat transfer coefficients τ′″₁.

When the smallest one of the differences between the four first estimated temperatures T″₁ₙ and the measured temperature is greater than the difference between the first estimated temperature T′₁ₙ and the measured temperature, four heat transfer coefficients τ″″₁ that are further approximate to the heat transfer coefficient τ′₁ are estimated to repeatedly carry out a series of calculations, such as calculations of first estimated temperatures T″″₁ₙ respectively based on the four heat transfer coefficients τ″″₁, or the heat transfer coefficient τ′₁ is input to the simulator.

In step S02, the heating condition of the object is set to the simulator by, for example, a user's input. Examples of the heating condition include the ambient temperature T_{wn} and the heating time.

In step S03, the simulator calculates the first estimated temperature T₁ₙ, which is the temperature of the central portion of the object, based on the various conditions set in step S01 and step S02. As the “central portion of the object” in this context, the central point P of the object or a portion close thereto can be employed, and the central point P of the object is employed in this embodiment. In this case, the central point temperature Tₚₙ of the object is used as the first estimated temperature T₁ₙ. As the central point P of the object, the center of mass of the object, that is, the center of gravity of the object can be employed. The position of the center of gravity of the object can be obtained by a known method.

The central point temperature Tₚₙ is calculated using the formula below, which derives from the ATS method, as aforementioned:

Tₚₙ=Tₚₙ₋₁+τ(T_{w}^{†}ₙ₋₁−Tₚₙ₋₁)

In step S04, the simulator calculates the second estimated temperature T₂ₙ as a temperature in a second area, based on the first estimated temperature T₁ₙ and the ambient temperature T_{wn}. In this embodiment, the second estimated temperature T₂ₙ is expressed as a simple average of the central point temperature Tₚₙ (equivalent to the first estimated temperature T₁ₙ) and the ambient temperature T_{wn}, and is specifically calculated using the formula below:

T₂ₙ=(Tₚₙ+T_{wn})/2

In step S05, the simulator calculates the third estimated temperature T₃ₙ, based on the first estimated temperature T₁ₙ, the second estimated temperature T₂ₙ, a ratio of a volume V_{AR1} of a first area AR1 to the volume of the object 100, and a ratio of a volume V_{AR2} of the second area AR2 to the volume of the object 100. Specifically, the third estimated temperature T₃ₙ is calculated as the volume average temperature Tₚ*ₙ between the first area AR1 and the second area AR2. As the first estimated temperature T₁ₙ, the central point temperature Tₚₙ is used as aforementioned. As the second estimated temperature T₂ₙ, the simple average of the central point temperature Tₚₙ and the ambient temperature T_{wn} is used as aforementioned. Thereby, the volume average temperature Tₚ*ₙ is calculated using the formula below, based on the central point temperature Tₚₙ and the ambient temperature T_{wn}.

Tₚ*ₙ=0.296Tₚₙ+0.704(T_{wn}+Tₚₙ)/2

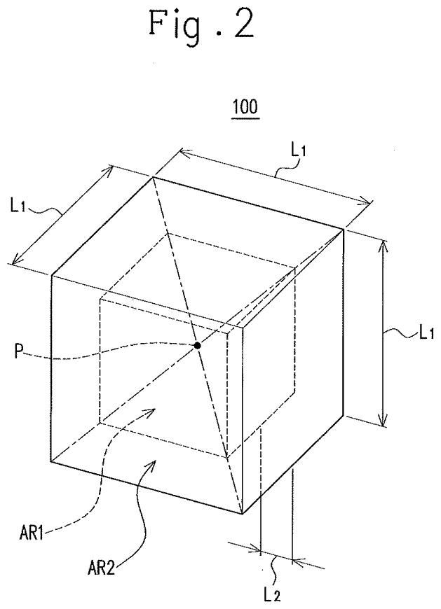

The object may have a cubic shape, a rectangular parallelepiped shape, and other shapes, and is assumed to have a cubic shape shown in FIG. 2 in this embodiment. The object 100 includes the first area AR1, which is an area positioned in the central portion of the object 100, and the second area AR2 surrounding the first area AR1. The object may be divided into two in any way as long as the first area AR1 is positioned in the central portion. In this embodiment, the object 100 is divided into the central portion and the surface portion, and the central portion is called the first area AR1 while the surface portion is called the second area AR2.

The formula above is derived as follows. Assume that the object 100 has the sides having a length of L₁, as shown in FIG. 2. Accordingly, a volume V of the object 100 is expressed as L₁³, and a surface area A of the object 100 is expressed as 6L₁². Where the distance from the surface of the first area AR1 to the surface of the second area AR2 is L₂, the distance L₂ corresponds to the surface thickness (V/A) of the object 100; thus, the distance L₂ is calculated as follows:

L₂=V/A=L₁/6

In this case, the volume V_{AR1} of the first area AR1 is calculated as follows:

V_{AR1}=(L₁−2·L₂)³=(⅔)³L₁³

The volume average temperature Tₚ*ₙ is obtained by adding the product of multiplication of the central point temperature Tₚₙ and the ratio of the volume V_{AR1} of the first area AR1 to the volume V of the object 100, to the product of multiplication of the simple average of the central point temperature Tₚₙ and the ambient temperature T_{wn} and the ratio of the volume V_{AR2} of the second area AR2 to the volume V of the object 100. Specifically, the volume average temperature Tₚ*ₙ is calculated using the formula below:

Tₚ*ₙ=Tₚₙ·V_{AR1}/V+((T_{wn}+Tₚₙ)/2)·(V·V_{AR1})/V

The formula below for calculating the volume average temperature Tₚ*ₙ is obtained by substituting (⅔)³L₁³ for V_{AR1} and L₁³ for V in the formula above:

Tₚ*n=(⅔)³Tₚₙ+(1−(⅔)³)·(T_{wn}+Tₚₙ)/2

The values in the formula above are rounded off to the third decimal place to obtain the formula below:

Tₚ*ₙ=0.296Tₚₙ+0.704(T_{wn}+Tₚₙ)/2

Performance of the simulation method according to this embodiment allows calculations of the first, second, and third estimated temperatures, as aforementioned. In the case where the object is, for example, retort food, the calculated first, second, and third estimated temperatures are used as indices for evaluating the heating condition of the food from a plurality of viewpoints, including the viewpoint of sterilization. Hereinafter, a description will be given on the case of evaluating the heating conditions of food using the first, second, and third estimated temperatures as the indices from two viewpoints, i.e., the viewpoint of sterilization and the viewpoint of protein degradation.

Whether or not a certain heating condition is suitable from the viewpoint of food sterilization is generally evaluated based on whether the temperature history of food being heated meets an F-value that is an integrated value of sterilization evaluation. Further, from the viewpoint of sterilization, it is preferable to evaluate the temperature of the central portion of food, which is least likely to be sterilized. Therefore, in order to evaluate the heating condition from the viewpoint of sterilization, the first estimated temperature history T₁ is obtained from the first estimated temperatures T₁ₙ to evaluate whether the first estimated temperature history T₁ is equivalent to the specified F-value.

Whether or not a certain heating condition is suitable from the viewpoint of protein degradation is evaluated based on, for example, whether the temperature history of food during the heating meets a C-value that is an integrated value of protein degradation evaluation expressed in a relation between temperature and time. Further, from the viewpoint of protein degradation, it is preferable to evaluate the temperature of the entire food during the heating. Therefore, in order to evaluate the heating condition from the viewpoint of protein degradation, the third estimated temperature history T₃ is obtained from the third estimated temperatures T₃ₙ to evaluate whether the third estimated temperature history T₃ meets the specified C value.

As above, the first estimated temperature T₁ₙ is used as the index for evaluating the heating condition from the viewpoint of sterilization, and the third estimated temperature T₃ₙ is used as the index for evaluating the heating condition from the viewpoint of protein degradation. Hereinafter, effects of this embodiment will be collectively described.

The first estimated temperature T₁ₙ that is calculated through the simulation method of this embodiment is suitable as an index for evaluating the heating condition from the viewpoint of sterilization. On the other hand, the second estimated temperature T₂ₙ is an temperature in the area surrounding the area of the central portion of the object, and thus preferable as an index for evaluating the heating condition from other viewpoints than sterilization. As above, the simulation method of this embodiment enables more accurate evaluation of the heating condition not only from the viewpoint of sterilization of the object but also from other viewpoints than sterilization.

In the simulation method of this embodiment, considering the ratios of the volumes V1 and V2 of the first and second areas AR1 and AR2, respectively, to the volume V of the object 100 are considered enables a more accurate temperature (approximate to the temperature of the object 100) to be obtained and a more accurate heating condition to be evaluated.

Use of the single temperature, namely the third estimated temperature T₃ₙ, allows the heating condition to be more easily evaluated than the case where a plurality of temperatures, i.e., the first and second estimated temperatures T₁ₙ and T₂ₙ are used.

The second estimated temperature T₂ₙ is calculated as the average of the first estimated temperature T₁ₙ and the ambient temperature T_{wn}, and thereby calculated more easily than the case where it is calculated using, for example, a complicated relational expression such as that of heat balance.

The simulation method of the present invention is not limited to the method of the aforementioned embodiment, but various modifications can be made without departing from the gist of the present invention.

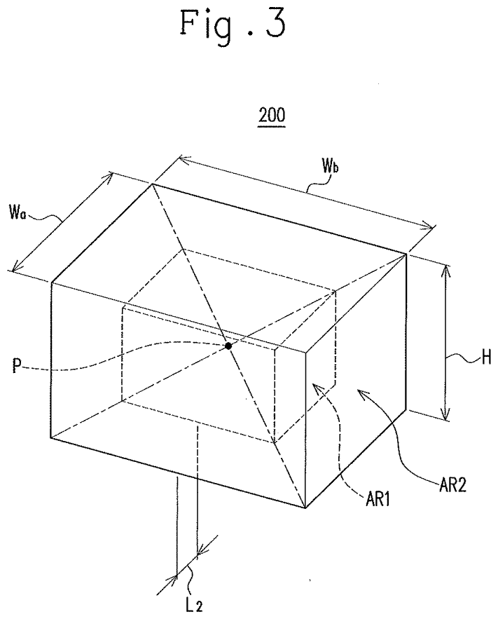

The aforementioned embodiment has been described by taking, for example, the case where the object is assumed to have a cubic shape, without limitation thereto. The object may be assumed to have, for example, a rectangular parallelepiped shape, a cylindrical shape, a spherical shape, and other shapes. When, as shown in FIG. 3, an object 200 is assumed to have a rectangular parallelepiped shape, the volume average temperature Tₚ*ₙ based on the central point temperature Tₚₙ and the ambient temperature T_{wn}, that is, the third estimated temperature T₃ₙ, can be calculated as follows.

Assume that the object 200 includes the first area AR1, which is the area of the central portion, and the second area AR2 surrounding the first area AR1. Assume that the second area AR2 is the surface portion of the object 200. Assume that, in the object 200, the length of a short side of the bottom surface is wₐ, the length of a long side of the bottom surface is w_{b}, and the height is H. In this case, the volume V of the object 200 is expressed as V=wₐ·w_{b}·H. The surface area A of the object 200 is expressed as 2 (wₐ·w_{b}+wₐ·H+w_{b}·H). Where the distance from the surface of the first area AR1 to the surface of the second area AR2 is L₂, the volume V_{AR1} of the first area AR1 is represented by the formula below:

V_{AR1}=(wₐ−2L₂)·(w_{b}−2L₂)·(H−2L₂)

The surface thickness (V/A) can be employed as the distance L₂; thus, the distance L₂ is calculated by the formula below:

L₂=V/A=wₐ·w_{b}·H/(wₐ·w_{b}+wₐ·H+w_{b}·H)

Further, the volume average temperature Tₚ*ₙ is represented by the formula below:

Tₚ*ₙ=Tₚₙ·V_{AR1}/V+((T_{wn}+Tₚₙ)/2)·(V−V_{AR1})/V

The volume average temperature Tₚ*ₙ is calculated based on the central point temperature Tₚₙ and the ambient temperature T_{wn}, by substituting the values of the volume V and the volume V_{AR1} into the formula above.

Obtaining the volume average temperature Tₚ*ₙ, that is, the third estimated temperature T₃ₙ as above enables calculation of an estimated temperature more appropriate for the shape of the object 200.

The aforementioned embodiment has been described by taking, for example, the case where the simple average of the central point temperature Tₚₙ and the ambient temperature T_{wn} is used as the second estimated temperature T₂ₙ, without limitation thereto. For example, another average may be used as the second estimated temperature T₂ₙ. Specifically, an average in consideration of the temperature gradient in the second area, an average in consideration of the shape or specific heat of the second area, an average in consideration of the volumes of the two areas obtained by arbitrarily dividing the second area into two, or the like, may be used as the second estimated temperature T₂ₙ. Use of such an average as the second estimated temperature T₂ₙ enables obtaining a temperature more approximate to the temperature of the actual object.

The aforementioned embodiment has been described by taking, for example, the case where the third estimated temperature T₃ₙ is calculated, and the third estimated temperature history T₃ obtained from the third estimated temperatures T₃ₙ is used as the index to evaluate the heating condition from other viewpoints than sterilization, without limitation thereto. For example, the first estimated temperature T₁ₙ and the second estimated temperature T₂ₙ may be used as the indices without calculating the third estimated temperature T₃ₙ to evaluate the heating condition from other viewpoints than sterilization. Use of the first estimated temperature T₁ₙ and the second estimated temperature T₂ₙ as the indices enables individual evaluations of the temperature histories in the first and second areas respectively, in contrast to the case where the third estimated temperature T₃ₙ is used, and thereby enables more appropriate evaluation of the heating condition approximate to the temperature history of the actual object.

The aforementioned embodiment has been described by taking, for example, the case where the object is divided into two to calculate the third estimated temperature, without limitation thereto. For example, the object may be divided into three or more areas to calculate the estimated temperature of each of the areas. Further, the estimated temperature history calculated from each estimated temperature may be used as the index to evaluate the heating condition. Use of the estimated temperature history in each of the three or more areas as the index enables evaluation of the heating condition more appropriate for the temperature history of the object.

The aforementioned embodiment has been described by taking, for example, the case where the first estimated temperature is calculated using the ATS method, without limitation thereto. For example, the first estimated temperature may be calculated using other methods such as Ball's formula method.

The simulator of the aforementioned embodiment may be an apparatus provided separately from an apparatus for heating the object, or may be integrated into the apparatus for heating the object.

The aforementioned embodiment has been described by taking, for example, the case where the object is retort food, without limitation thereto. For example, the object may be other packed food such as canned food, or an object other than food, including, for example, a medicinal product and a medical device such as a syringe.

The aforementioned embodiment has been described by taking, for example, the case where the object is food and the heating condition under which the object is heated is evaluated from two viewpoints, namely: sterilization and protein degradation, without limitation thereto. For example, in the case where the object is food, examples of the viewpoints for evaluation other than sterilization include vitamin degradation, enzyme degradation, and deterioration in texture or color. In the case where the object is a medicinal product, a medical device, or the like, examples of the viewpoints for evaluation other than sterilization include deterioration of components contained in the medicinal product and materials forming the medical device.

As described above, the simulation method according to the present invention includes: a step of setting a heating condition of an object; a step of calculating a first estimated temperature as a temperature of a central portion of the object, based on the heating condition; and a step of calculating a second estimated temperature as a temperature in a second area that surrounds a first area positioned in the central portion of the object, based on the first estimated temperature.

The first and second estimated temperatures calculated using the aforementioned simulation method are used for evaluating the heating condition from the viewpoint of sterilization or from other viewpoints than sterilization. The first estimated temperature is an estimated temperature of the central portion of the object, and thus suitable as an index for evaluating the heating condition from the viewpoint of sterilization. The second estimated temperature is a temperature in the area surrounding the central area of the object, and thus preferable as an index for evaluating the heating condition from other viewpoints than sterilization. As above, the simulation method capable of more accurate evaluation of the heating condition not only from the viewpoint of sterilization of the object but also from other viewpoints than sterilization can be provided.

As one aspect of the simulation method according to the present invention, the simulation method may further include a step of evaluating a temperature history of the object, based on the first estimated temperature, the second estimated temperature, a ratio of a volume of the first area to a volume of the object, and a ratio of a volume of the second area to the volume of the object.

Consideration of the ratio of the volume of each of the first and second areas to the volume of the object as above enables obtaining a more accurate temperature (approximate to the temperature of the actual object), and the simulation method capable of more accurate evaluation of the heating condition can be provided.

Further, as one aspect of the simulation method according to the present invention, in the step of evaluating the temperature history of the object, a third estimated temperature may be calculated based on the first estimated temperature, the second estimated temperature, the ratio of the volume of the first area to the volume of the object, and the ratio of the volume of the second area to the volume of the object, to evaluate the temperature history of the object using the third estimated temperature.

Use of the single temperature, namely the third estimated temperature, as above enables providing the simulation method capable of evaluating the heating condition more easily than the case where a plurality of temperatures, namely the first and second estimated temperatures, are used.

As one aspect of the simulation method according to the present invention, the heating condition may include an ambient temperature and a heating time of the object, and the second estimated temperature may be an average of the first estimated temperature of the object and the ambient temperature.

As above, the second estimated temperature is the average of the first estimated temperature and the ambient temperature, and thus the simulation method capable of calculating the second estimated temperature more easily can be provided than the case where it is calculated using a complicated relational expression such as that of heat balance.

The simulation program according to the present invention is a simulation program for causing an arithmetic unit to execute calculation of an estimated temperature of an object to be heated, the simulation program causing the arithmetic unit to execute: a step of receiving a setting of a heating condition of the object; a step of calculating a first estimated temperature as a temperature of a central portion of the object, based on the heating condition; and a step of calculating a second estimated temperature as a temperature in a second area that surrounds a first area positioned in the central portion of the object, based on the first estimated temperature.

The first estimated temperature is an estimated temperature of the central portion of the object and thus suitable as an index for evaluating the heating condition from the viewpoint of sterilization while the second estimated temperature is a temperature in the area surrounding the central area of the object and thus preferable as an index for evaluating the heating condition from other viewpoints than sterilization. As above, the simulation program capable of evaluating the heating condition not only from the viewpoint of sterilization but also from other viewpoints than sterilization can be provided.

The simulator according to the present invention includes a storage medium having the simulation program stored therein, and is configured to cause the arithmetic unit to run the simulation program.

The first estimated temperature is an estimated temperature of the central portion of the object and thus suitable as an index for evaluating the heating condition from the viewpoint of sterilization while the second estimated temperature is a temperature in the area surrounding the central area of the object and thus preferable as an index for evaluating the heating condition from other viewpoints than sterilization. As above, the simulator capable of evaluating the heating condition not only from the viewpoint of sterilization but also from other viewpoints than sterilization can be provided.

### INDUSTRIAL APPLICABILITY

The simulation method of the present invention is applicable to calculating temperature change resulting from heating of retort food, canned food, medicinal products, medical devices, or the like.

### REFERENCE SIGNS LIST

100: Object
AR1: First area
AR2: Second area
P: Central point

1. A simulation method, comprising:
a step of setting a heating condition of an object;
a step of calculating a first estimated temperature as a temperature of a central portion of the object, based on the heating condition; and
a step of calculating a second estimated temperature as a temperature in a second area that surrounds a first area positioned in the central portion of the object, based on the first estimated temperature.

2. The simulation method according to claim 1, further comprising a step of evaluating a temperature history of the object, based on the first estimated temperature, the second estimated temperature, a ratio of a volume of the first area to a volume of the object, and a ratio of a volume of the second area to the volume of the object.

3. A simulation method according to claim 2, wherein
in the step of evaluating the temperature history of the object, a third estimated temperature is calculated based on the first estimated temperature, the second estimated temperature, the ratio of the volume of the first area to the volume of the object, and the ratio of the volume of the second area to the volume of the object, to evaluate the temperature history of the object using the third estimated temperature.

4. The simulation method according to claim 1, wherein
the heating condition includes an ambient temperature and a heating time of the object, and
the second estimated temperature is an average of the first estimated temperature of the object and the ambient temperature.

5. A simulation program for causing an arithmetic unit to execute calculation of an estimated temperature of an object to be heated, the simulation program causing the arithmetic unit to execute:
a step of receiving a setting of a heating condition of the object;
a step of calculating a first estimated temperature as a temperature of a central portion of the object, based on the heating condition; and
a step of calculating a second estimated temperature as a temperature in a second area that surrounds a first area positioned in the central portion of the object, based on the first estimated temperature.

6. A simulator comprising a storage medium having the simulation program according to claim 5 stored therein, and configured to cause the arithmetic unit to run the simulation program.

7. The simulation method according to claim 2, wherein
the heating condition includes an ambient temperature and a heating time of the object, and
the second estimated temperature is an average of the first estimated temperature of the object and the ambient temperature.

8. The simulation method according to claim 3, wherein
the heating condition includes an ambient temperature and a heating time of the object, and
the second estimated temperature is an average of the first estimated temperature of the object and the ambient temperature.
